(19) United States
(12) Patent Application Publication
(10) Pub. No.: US 2015/0252385 A1
(43) Pub. Date: Sep. 10, 2015

(54) Methods for Producing Recombinant Proteins

(71) Applicant: UCB Biopharma SPRL, Brussels (BE)

(72) Inventor: Katharine Lacy Cain, Slough (GB)

(21) Appl. No.: 14/431,855

(22) PCT Filed: Sep. 30, 2013
(86) PCT No.: PCT/EP2013/070317

§ 371 Date: Mar. 27, 2015

(30) Foreign Application Priority Data
Sep. 28, 2012 (GB) 1217398.5
Jun. 18, 2013 (GB) 1310868.3

(51) Int. Cl. C12N 15/90; C07K 16/00; C12N 9/14; C12N 15/85

(52) U.S. Cl. CPC C12N 15/90; C12N 15/85; C07K 16/00; C12N 9/14; C07K 2317/14; C12Y 306/04; C12Y 306/01

(57) ABSTRACT

The present disclosure relates to a host cell transfected with a NHEJ protein or a functional fragment thereof or a polynucleotide encoding the same, wherein said polynucleotide sequence is under the control of a suitable promoter and the host cell is also transfected with an expression cassette comprising a polynucleotide sequence encoding at least one protein of interest, methods of preparing the host cells, plasmids and intermediates employed in the preparation of the same and use of the host cells to express to protein.

The present disclosure relates to a method of increasing the number of host cells stably transfected with a DNA sequence encoding a protein of interest and capable of expressing the latter. The disclosure also relates to host cells prepared employing the method herein and polynucleotide sequences employed in the method, such as RNA or DNA, in particular plasmid DNA.

### BACKGROUND

“Cultivated mammalian cells have become the dominant system for the production of recombinant proteins for clinical applications because of their capacity for proper protein folding, assembly and post-translational modification. Thus, the quality and efficacy of a protein can be superior when expressed in mammalian cells versus other hosts such as bacteria, plants and yeast.

Today about 60-70% of all recombinant protein pharmaceuticals are produced in mammalian cells. In addition, estimates speak of several hundred clinical candidate proteins currently in company pipelines. Many of these proteins are expressed in immortalized Chinese hamster ovary (CHO) cells, but other cell lines, such as those derived from mouse myeloma (NSO), baby hamster kidney (BHK), human embryo kidney (HEK-293) and human retinal cells have gained regulatory approval for recombinant protein production. Although the majority of today's mammalian cell culture processes for biopharmaceutical production are based on cells that are cultivated in suspension, the diversity of actual manufacturing approaches is considerable.

Initially, the recombinant gene with the necessary transcriptional regulatory elements is transferred to the cells. In addition, a second gene is transferred that confers to recipient cells a selective advantage. In the presence of the selection agent, which is applied a few days after gene transfer, only those cells that express the selector gene survive. The most popular genes for selection are dihydrofolate reductase (DHFR), an enzyme involved in nucleotide metabolism, and glutamine synthetase (GS). In both cases, selection occurs in the absence of the appropriate metabolite (hypoxantine and thymidine, in the case of DHFR, glutamine in the case of GS), preventing growth of non-transformed cells. In general, for efficient expression of the recombinant protein, it is not important whether the biopharmaceutical encoding gene and selector genes are on the same plasmid or not.

Following selection, survivors are transferred as single cells to a second cultivation vessel, and the cultures are expanded to produce clonal populations. Eventually, individual clones are evaluated for recombinant protein expression, with the highest producers being retained for further cultivation and analysis. From these candidates, one cell line with the appropriate growth and productivity characteristics is chosen for production of the recombinant protein. A cultivation process is then established that is determined by the production needs. So far, all mammalian recombinant therapeutics are naturally secreted proteins or have been developed from gene constructs that mediate protein secretion.”

The above is an extract from a Nature review paper published in 2004 (Florian Wurm Nature Biotechnology Vol 22 Number 11 Nov. 2004, page 1393-1398). In 2012, the processes are essentially unchanged from the processes described above, which was published in 2004.

This Nature Biotechnology review discusses the disadvantages of random insertion into the host cell genome and they state that in fact little or no transgene expression is obtained where the transgene is inserted into to certain parts of the cell's genome, such as the tightly packed from of DNA known as heterochromatin.

Even where heterologous genes are inserted into permissive areas of the genome, for example the less tightly packed DNA known as euchromatin, neighbouring genes may still exert a negative influence and thus the transgene may be inactivated.

The Nature review paper goes on to say: Several strategies have been developed to overcome the negative position effects of random integration. Protective cis-regulatory elements include insulators, boundary elements, scaffold/matrix attachment regions, ubiquitous chromatin opening elements and conserved anti-repressor elements. Flanking transgenes with these elements reduces the effects of heterochromatin and allows stable expression of the transgene. Another way to inhibit silencing is to block deacetylation of histones using butyrate. Targeting transgene integration to transcriptionally active regions of the genome is another possible strategy to avoid position effects, and presentations at conferences in recent years have hinted at the use of gene targeting in industry.”

Some in the field have focused on targeted integration, sometimes referred to homologous recombination, but this requires good knowledge of the host cells genome and these events are relatively rare without further assistance from enzymes.

The generation of suitable clones for manufacturing purposes often requires extensive screening and laborious searching, for example it may require analysis of 1,500 clones to identify one that is suitable for expressing the protein of interest.

The present inventors have surprisingly established that co-transfection of a host cell with a DNA sequence encoding a protein of interest and a NHEJ protein for example Ku70, Ku80, a combination thereof or a functional fragment thereof or sequences encoding same results in the generation of many more cells capable of expressing the protein of interest.

### SUMMARY OF THE DISCLOSURE

Thus there is provided a host cell transfected with an NHEJ protein or a functional fragment thereof, for example Ku70, Ku80, a combination thereof or a polynucleotide sequence encoding same, wherein said polynucleotide sequence is under the control of a suitable promoter and the host cell is also transfected with an expression cassette comprising a DNA or RNA sequence encoding at least one protein of interest.

In one embodiment there is provided a host cell transfected with a polynucleotide sequence encoding an NHEJ protein or a functional fragment thereof, for example Ku70, Ku80, a combination thereof, wherein said DNA sequence is under the control of a suitable promoter and the host cell is also transfected with an expression cassette comprising a DNA sequence encoding a protein of interest.

There is also provided plasmid DNA comprising a sequence encoding a NHEJ protein or a functional fragment thereof, for example Ku70, Ku80, a combination thereof or a functional fragment thereof wherein said DNA sequence is under the control of a suitable promoter.

The present disclosure further includes a method of generating a host capable of expressing a protein of interest encoded by a DNA sequence comprising the step of co-transfecting the cell with:
a. Ku70, Ku80, a combination thereof or a functional fragment thereof, or a polynucleotide sequence encoding same wherein said polynucleotide sequence is under the control of a suitable promoter, and
b. an expression cassette comprising a DNA or RNA sequence encoding a protein of interest.

In one embodiment there is provided a method of generating a host cell capable of expressing a protein of interest encoded by a DNA sequence said method comprising the step of co-transfecting the cell with:
a. a polynucleotide sequence encoding an NHEJ protein or a functional fragment thereof, for example Ku70, Ku80, a combination thereof or a functional fragment thereof, wherein said polynucleotide sequence is under the control of a suitable promoter, and
b. an expression cassette comprising a DNA sequence encoding a protein of interest.

Advantageously employing an NHEJ protein or a functional fragment thereof, for example Ku70 and/or Ku80 encoding polynucleotide seems to increase the number of cells obtained that are capable of expressing the protein of interest. This in turn increases the probability of identifying a suitable stable cell-line for expressing the protein.

The present method significantly reduces the amount of effort and resource required to isolate a suitable manufacturing clone, for example 25 to 75% fewer clones may be analysed because the numbers of protein expressing clones are greatly increased employing the method.

### DETAILED DESCRIPTION OF THE DISCLOSURE

Non-homologous end joining (NHEJ) is a pathway that repairs double-strand breaks in DNA. NHEJ is referred to as “non-homologous” because the break ends are directly ligated without the need for a homologous template, in contrast to homologous recombination, which requires a homologous sequence to guide repair.

NHEJ typically utilizes short homologous DNA sequences called microhomologies to guide repair. These microhomologies are often present in single-stranded overhangs on the ends of double-strand breaks.

NHEJ is conserved throughout all kingdoms of life and is the predominant double-strand break repair pathway in mammalian cells. In budding yeast (Saccharomyces cerevisiae), however, homologous recombination dominates when the organism is grown under common laboratory conditions.

A NHEJ protein is one involved in or which facilitates NHEJ processes.

In one embodiment the NHEJ protein is employed in a purified form. In one embodiment the NHEJ protein comprises a purification tag, such as a his-tag to assist in the purification process.

A host cell as employed herein is a cell suitable for or capable of expressing a protein of interest after effective transfection has been accomplished. Of course expression may require appropriate functional elements such as promoters and the like.

In one embodiment the host cell is prokaryotic, for example bacterial, such E. coli.

In one embodiment the host cell is a eukaryotic cell, for example a yeast cell, an insect cell, or an animal cell such as a mammalian cell. In one embodiment the host cell is an animal cell, such as a mammalian cell, for example selected from Chinese hamster ovary (CHO) cells, but other cell lines, such as those derived from mouse myeloma (NSO), baby hamster kidney (BHK), human embryo kidney (HEK-293) and human retinal cells may be employed.

An alternative mammalian cell is a vero cell.

Non-mammalian cells lines include yeasts such as Pichia or bacterial cells lines such as E. coli.

Transfection as employed herein refers to is the process of deliberately introducing nucleic acids or polypeptides, such as proteins into a cell. Genetic material, such as supercoiled plasmid DNA, linear DNA or siRNA constructs, may be transfected. In one embodiment the term is used for non-viral methods in eukaryotic cells.

Transfection of animal cells typically involves opening transient pores or “holes” in the cell membrane, to allow the uptake of material. Transfection can be carried out using calcium phosphate, by electroporation, or by mixing a cationic lipid with the material to produce liposomes, which fuse with the cell membrane and deposit their cargo inside.

Ku70 is a protein that, in humans, is encoded by the XRCC6 gene. Together, Ku70 and Ku80 make up the Ku heterodimer, which binds to DNA double-strand break ends and is required for the non-homologous end joining (NHEJ) pathway of DNA repair. It is also required for V(D)J recombination, which utilizes the NHEJ pathway to promote antigen diversity in the mammalian humoral immune system.

In addition to its role in NHEJ, Ku is also required for telomere length maintenance and sub-telomeric gene silencing. The human protein Ku70 has the UniProt number P12956 and the murine protein has the number P23475.

A functional fragment of the Ku70 protein as employed herein refers to a fragment of the protein that retains the essential biological function of the protein, in particular the NHEJ function.

Ku80 is a protein that, in humans, is encoded by the XRCC5 gene. The human protein has the UniProt number P13010 and the murine protein has the number P27641.

A functional fragment of the Ku80 protein as employed herein refers to a fragment of the protein that retains the essential biological function of the protein, in particular the NHEJ function.

Functional fragment in the context of a polynucleotide refers to a polynucleotide, which encodes a functional fragment of a protein, for example as described herein.

In one embodiment the Ku encoding polynucleotide(s) or proteins employed are independently selected from the species hamster, mouse, rat, human or other species where the gene/protein has the same function. In one embodiment the polynucleotide or protein employed has >50%, 60%, 70%, 80%, 90%, such as 95% sequence identity at the nucleotide/protein level.

In one embodiment a polynucleotide employed in the present disclosure hybridises under stringent conditions to a polynucleotide encoding a Ku protein or combination of Ku proteins.

It will be appreciated that any suitable NHEJ protein from any suitable species may be used depending on the nature of the host cell line employed. In one example the NHEJ protein is human. In one example the NHEJ protein is human Ku70 alone or in combination with human Ku80. In one example the NHEJ protein is human Ku80 alone or in combination with human Ku70. In one example the NHEJ protein is hamster Ku70 alone or in combination with hamster Ku80. In one example the NHEJ protein is hamster Ku80 alone or in combination with hamster Ku70.

In one embodiment an alternative gene to Ku 70 or 80 with NHEJ function is employed.

Prokaryotic homologs of DNA-end-binding proteins are discussed in Aravind and Koonin, Genome Research 2001 Aug. 11(8) 1365-1374.

In one embodiment a polypeptide employed encodes a protein of eukaryotic origin, such as a mammalian protein.

A promoter as employed herein refers to a region of DNA that initiates transcription of a particular gene. Promoters are generally located near the genes they transcribe, on the same strand and upstream (towards the 5′ region of the sense strand).

Suitable promoter as employed herein refers to a promoter that is fit for the purpose of initiating transcription of the target polynucleotide in the relevant genetic environment.

In one embodiment the promoter is a viral promoter, for example a strong promoter such as a CMV promoter, for example a Tn7 promoter, a lac promoter, a tac promoter or an inducible promoter such a tetracycline responsive promoter, an alcohol inducible promoter or a glucose inducible promoter.

Tetracycline-controlled transcriptional activation is a method of inducible expression where transcription is reversibly turned on or off in the presence of the antibiotic tetracycline or one of its derivatives (e.g. doxycycline). In nature, the Pₜₑₜ promoter expresses TetR, the repressor, and TetA, the protein that pumps tetracycline antibiotic out of the cell

In one embodiment a polynucleotide sequence encoding an NHEJ protein or a functional fragment thereof, for example Ku70, Ku80, a combination thereof or a functional fragment thereof, is provided under the control of a suitable promoter, for example as described herein such as an inducible promoter.

Polynucleotide as employed herein refers to is a biopolymer composed of 13 or more nucleotide monomers covalently bonded in a chain. DNA (deoxyribonucleic acid) and RNA (ribonucleic acid) are examples of polynucleotides with distinct biological function.

In one embodiment the polynucleotide is DNA, for example plasmid DNA, in circular (supercoiled, closed or relaxed) form or linear form.

In one embodiment the polynucleotide is RNA.

An expression cassette directs the cell's machinery to make RNA and protein. Some expression cassettes are designed for modular cloning of protein-encoding sequences so that the same cassette can easily be altered to make different proteins. In one embodiment the cassette comprises one or more genes and the sequences for controlling their expression, for example it comprises at least three components namely: a promoter sequence, an open reading frame, and a 3′ untranslated region that, in eukaryotes, usually contains a polyadenylation site.

In one embodiment the protein of interest is an antibody or a binding fragment thereof.

The term ‘antibody’ as used herein refers to an immunoglobulin comprising a complete antibody molecule having full length heavy and light chains and bi-specific molecules comprising the same. For example, the antibodies may be IgA, IgD, IgE, IgG or IgM, in particular, human IgG1, IgG2, IgG3or IgG4 isotypes.

Binding fragment thereof refers to a fragment of an antibody capable of binding the polypeptide to which it is specific and such fragments include, but are not limited to Fab, modified Fab, Fab′, modified Fab′, F(ab′)₂, Fv, Fab-Fv, Fab-dsFv, single domain antibodies (e.g. VH or VL or VHH), scFv, bi, tri or tetra-valent antibodies, Bis-scFv, diabodies, triabodies, tetrabodies and epitope-binding fragments of any of the above (see for example Holliger and Hudson, 2005, Nature Biotech. 23(9):1126-1136; Adair and Lawson, 2005, Drug Design Reviews—Online 2(3), 209-217). The methods for creating and manufacturing these antibody fragments are well known in the art (see for example Verma et al., 1998, Journal of Immunological Methods, 216, 165-181). Other antibody fragments for use in the present invention include the Fab and Fab′ fragments described in International patent applications WO2005/003169, WO2005/003170 and WO2005/003171.

Multi-valent antibodies may comprise multiple specificities e.g bispecific or may be monospecific (see for example WO92/22853 and WO05/113605). Further examples include the bispecific antibodies described in WO2009/040562 and WO2010/035012

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al. (supra)”). This numbering system is used in the present specification except where otherwise indicated.

The Kabat residue designations do not always correspond directly with the linear numbering of the amino acid residues. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence.

The CDRs of the heavy chain variable domain are located at residues 31-35 (CDR-H1), residues 50-65 (CDR-H2) and residues 95-102 (CDR-H3) according to the Kabat numbering system. However, according to Chothia (Chothia, C. and Lesk, A. M. J. Mol. Biol., 196, 901-917 (1987)), the loop equivalent to CDR-H1 extends from residue 26 to residue 32. Thus unless indicated otherwise ‘CDR-H1’ as employed herein is intended to refer to residues 26 to 35, as described by a combination of the Kabat numbering system and Chothia's topological loop definition.

The CDRs of the light chain variable domain are located at residues 24-34 (CDR-L1), residues 50-56 (CDR-L2) and residues 89-97 (CDR-L3) according to the Kabat numbering system.

In one embodiment the antibody or binding fragment thereof is monoclonal or polyclonal, such as monoclonal.

Monoclonal antibodies may be prepared by any method known in the art such as the hybridoma technique (Kohler & Milstein, 1975, Nature, 256:495-497), the trioma technique, the human B-cell hybridoma technique (Kozbor et al., 1983, Immunology Today, 4:72) and the EBV-hybridoma technique (Cole et al., Monoclonal Antibodies and Cancer Therapy, pp 77-96, Alan R Liss, Inc., 1985)

Monoclonal antibodies may also be isolated with screening, for example based on a analysing the variable-gene repertoire of plasma cells see for example Reddy et al., Nature Biotechnology 28:965-969 (2010)).

Antibodies or binding fragment thereof for use in the invention may also be generated using single lymphocyte antibody methods by cloning and expressing immunoglobulin variable region cDNAs generated from single lymphocytes selected for the production of specific antibodies by for example the methods described by Babcook, J. et al., 1996, Proc. Natl. Acad. Sci. USA 93(15):7843-78481; WO92/02551; WO2004/051268 and International Patent Application number WO2004/106377.

The antibodies or binding fragment thereof for use in the present invention can also be generated using various phage display methods known in the art and include those disclosed by Brinkman et al. (in J. Immunol Methods, 1995, 182: 41-50), Ames et al. (J. Immunol. Methods, 1995, 184:177-186), Kettleborough et al. (Eur. J. Immunol 1994, 24:952-958), Persic et al. (Gene, 1997 187 9-18), Burton et al. (Advances in Immunology, 1994, 57:191-280) and WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

The antibodies or binding fragment thereof for use in the present invention can also be generated using various yeast display methods known in the art, for example as described in J Immunol Methods 2004 July; 290(1-2): 69-80.

The antibodies or binding fragment thereof for use in the present invention can also be generated using various mammalian cell surface display methods, for example as described in MAbs 2010 Sep.-Oct. 2(5): 508-518.

In one embodiment the antibody or binding fragment thereof is humanised.

Humanised antibodies (which include CDR-grafted antibodies) are antibody molecules having one or more complementarity determining regions (CDRs) from a non-human species and a framework region from a human immunoglobulin molecule (see, e.g. U.S. Pat. No. 5,585,089; WO91/09967). It will be appreciated that it may only be necessary to transfer the specificity determining residues of the CDRs rather than the entire CDR (see for example, Kashmiri et al., 2005, Methods, 36, 25-34). Humanised antibodies may optionally further comprise one or more framework residues derived from the non-human species from which the CDRs were derived.

In one embodiment the antibody or binding fragment thereof is chimeric.

Chimeric antibodies are those antibodies encoded by immunoglobulin genes that have been genetically engineered so that the light and heavy chain genes are composed of immunoglobulin gene segments belonging to different species.

Fully human antibodies are those antibodies in which the variable regions and the constant regions (where present) of both the heavy and the light chains are all of human origin, or substantially identical to sequences of human origin, not necessarily from the same antibody. Examples of fully human antibodies may include antibodies produced for example by the phage display methods described above and antibodies produced by mice in which the murine immunoglobulin variable and constant region genes have been replaced by their human counterparts e.g. as described in general terms in EP0546073 B1, U.S. Pat. No. 5,545,806, U.S. Pat. No. 5,569,825, U.S. Pat. No. 5,625,126, U.S. Pat. No. 5,633,425, U.S. Pat. No. 5,661,016, U.S. Pat. No. 5,770,429, EP 0438474 B1 and EPO463151 B1.

Fully human antibodies can also be prepared in transgenic animals, for example transgenic mice, cows, pigs, sheep, donkeys and camelids, such as llama's and camels. See for example the review in Nature Biotechnology 23, 1117-1125 (2005).

In one example the antibodies or binding fragments thereof for use in the present invention may be derived from a camelid, such as a camel or llama. Camelids possess a functional class of antibodies devoid of light chains, referred to as heavy chain antibodies (Hamers et al., 1993, Nature, 363, 446-448; Muyldermans, et al., 2001, Trends. Biochem. Sci. 26, 230-235). The antigen-combining site of these heavy-chain antibodies is limited to only three hypervariable loops (H1-H3) provided by the N-terminal variable domain (VHH). The first crystal structures of VHHs revealed that the H1 and H2 loops are not restricted to the known canonical structure classes defined for conventional antibodies (Decanniere, et al., 2000, J. Mol. Biol, 300, 83-91). The H3 loops of VHHs are on average longer than those of conventional antibodies (Nguyen et al., 2001, Adv. Immunol, 79, 261-296). A large fraction of dromedary heavy chain antibodies have a preference for binding into active sites of enzymes against which they are raised (Lauwereys et al., 1998, EMBO J, 17, 3512-3520). In one case, the H3 loop was shown to protrude from the remaining paratope and insert in the active site of the hen egg white lysozyme (Desmyter et al., 1996, Nat. Struct. Biol. 3, 803-811). Accordingly, whilst clefts on protein surfaces are often avoided by conventional antibodies, heavy-chain antibodies of camelids have been demonstrated to be capable of entering enzyme active sites, largely due to the compact prolate shape of VHH formed by the H3 loop (De Genst et al., 2006, PNAS, 103, 12, 4586-4591 and WO97049805).

In one example the antibodies or binding fragments thereof for use in the present invention may be derived from a cartilaginous fish, such as a shark. Cartilaginous fish (sharks, skates, rays and chimeras) possess an atypical immunoglobulin isotype known as IgNAR. IgNAR is an H-chain homodimer that does not associate with light chain. Each H chain has one variable and five constant domains. IgNAR V domains (or V-NAR domains) carry a number of non-canonical cysteines that enable classification into two closely related subtypes, I and II. Type II V regions have an additional cysteine in CDRs 1 and 3 which have been proposed to form a domain-constraining disulphide bond, akin to those observed in camelid VHH domains. The CDR3 would then adopt a more extended conformation and protrude from the antibody framework akin to the camelid VHH. Indeed, like the VHH domains described above, certain IgNAR CDR3 residues have also been demonstrated to be capable of binding in the hen egg white lysozyme active site (Stanfield et al., 2004, Science, 305, 1770-1773.

Examples of methods of producing VHH and IgNAR V domains are described in for example, Lauwereys et al, 1998, EMBO J. 1998, 17(13), 3512-20; Liu et al., 2007, BMC Biotechnol., 7, 78; Saerens et al., 2004, J. Biol. Chem., 279 (5), 51965-72.

The antibodies or binding fragments thereof for use in the present invention may IgY antibodies or derivatives therefrom. IgY antibodies are generally produced by certain avian species, for example chickens. See for example Larson et al Poult Sci 1993 October; 72(10): 1807-12 and Davidson et al (2008) Avian Immunology Academic Press page 418.

In one embodiment the Ku70 and/or Ku 80 is transfected into the host cell and the DNA encoding a protein of interest is transfected into the cell on a separate plasmid or plasmids. This allows the DNA encoding the protein of interest to be stably integrated into the genome of the host cell, and in one embodiment the Ku70 and/or Ku80 may remain, transiently transfected. This may be desirable from a regulatory perspective.

In one embodiment the Ku70 and/or Ku80 are transfected into the cell on the same plasmid.

In one embodiment the Ku70 and/or Ku80 are transfected into the cell on separate plasmids.

In one embodiment the DNA encoding a protein of interest is on a plasmid which does not comprise a polynucleotide encoding the Ku70 and/or Ku80 protein.

In one embodiment the DNA encoding a protein of interest is on a plasmid and further comprises a polynucleotide encoding the Ku70 and/or Ku80 protein.

It may be advantageous to employ both Ku70 and Ku80 elements because this may provide transfectants (clones) expressing higher levels of the protein of interest.

In one embodiment the polynucleotide encoding the Ku70 and/or Ku80 is provided as RNA, for example linear RNA fragments and for example capable of translation after introduction into the cell.

In one embodiment the polynucleotide such as DNA and/or RNA is naked.

In one embodiment the polynucleotide is linear, for example linear DNA and/or RNA.

In one embodiment the polynucleotide is supercoiled, for example supercoiled DNA.

In one embodiment the polynucleotide, such as DNA and/or RNA is introduced into the cell employing one or more reagents, for example calcium phosphate or a cationic lipid.

The amount of Ku70 encoding polynucleotide transfected into the cell may be in the range 1 to 100 μg, for example 10 to 50 μg, such as 20 to 40 μg.

The amount of Ku80 encoding polynucleotide transfected into the cell may be in the range 1 to 100 μg, for example 10 to 50 μg, such as 20 to 40 μg.

The amount of Ku70 and Ku80 encoding polynucleotide transfected into the cell may be in the range 1 to 100 μg, for example 10 to 50 μg, such as 20 to 40 μg.

In one embodiment the amount of DNA encoding a protein of interest that is transfected into the cell is in the range 1 to 100 μg, such as 10 to 30 μg, in particular 20 μg.

In one embodiment the plasmid comprising the DNA encoding a protein of interest further comprises a selection marker, for example antibiotic resistance marker, dihydrofolate reductase or glutamine synthetase.

In one embodiment a host cell comprising the elements of the invention described herein further comprises a selection marker, for example antibiotic resistance marker, dihydrofolate reductase or glutamine synthetase.

In one embodiment the DNA encoding the protein of interest is stably integrated into the genome of the host cell.

In one embodiment the DNA encoding the protein of interest is transiently transfected in the host cell, for example as plasmid DNA.

In one embodiment the polynucleotide encoding a NHEJ protein such as a Ku protein or proteins is stably integrated in the host cell genome.

In one embodiment there is provided a host cell according to the present disclosure wherein the polynucleotide sequence encoding Ku70, Ku80, a combination thereof or a functional fragment thereof is transiently transfected into the cell.

It will be appreciated that the polynucleotide encoding a protein of interest and the polynucleotide encoding the NHEJ protein or proteins, such as the Ku protein or proteins can be transfected into the host cell:
at the same time, or
the polypeptide encoding the protein of interest can be transfected first followed by transfection of the polynucleotide encoding a Ku protein or proteins, or
the polynucleotide encoding a Ku protein or proteins can be transfected first followed by transfection of the polynucleotide encoding the protein of interest.

Transfection before (first as described above) includes for example transfection 2 to 48 hours before the subsequent transfection.

Thus there can be a temporal difference for the point at which transfection of the different components employed, occurs.

The disclosure further provides plasmid DNA comprising a sequence encoding Ku70, Ku80, a combination thereof or a functional fragment thereof wherein said DNA sequence is under the control of a suitable promoter.

In one embodiment the plasmid DNA further comprises a DNA encoding a protein of interest, for example wherein the protein of interest is an antibody or a binding fragment thereof.

In one embodiment the plasmid DNA described herein is circular.

In one embodiment the plasmid DNA described herein is linear. Linear plasmid DNA refers to DNA which is derived from a plasmid, for example by cutting circular plasmid DNA with one or more enzymes, such as restriction enzymes, to provide the DNA in a linear form. The linear form of the plasmid DNA will generally comprise one or more elements which are characteristic of a plasmid, for example origin of replication or similar.

In one embodiment the DNA is a PCR fragment. PCR fragment are identifiable in that they comprise the primer sequences used to identify the fragment.

In one embodiment the DNA is a restriction fragment i.e. a fragment provided by cleavage with one or more restriction enzymes.

In a further aspect there is provided a method of generating a host capable of expressing a protein of interest encoded by a DNA sequence comprising the step of co-transfecting the cell with:
a. a polynucleotide sequence encoding Ku70, Ku80, a combination thereof or a functional fragment thereof, wherein said polynucleotide sequence is under the control of a suitable promoter, and
b. an expression cassette comprising a DNA sequence encoding a protein of interest and optionally a selection marker.

In one embodiment the method employs a polynucleotide encoding Ku 70 or a functional fragment thereof.

In one embodiment the method employs a polynucleotide encoding Ku 80 or a functional fragment thereof.

In one embodiment the method employs a polynucleotide or polynucleotides independently encoding Ku 70 or a functional fragment thereof and Ku 80 or a functional fragment thereof. Where there is a single polynucleotide sequence clearly it will encode both proteins/fragments.

In one embodiment there is provided a method according the disclosure herein wherein the DNA sequence encoding Ku70, Ku80, a combination thereof or a functional fragment thereof is provided in the form of a plasmid.

In one embodiment the plasmid employed in the method is described supra.

In one embodiment there is provided a method wherein the expression cassette comprising a DNA sequence encoding a protein of interest in step b) is provided on a plasmid or plasmids.

In one embodiment there is provided a method as described herein, wherein the polynucleotide sequence encoding Ku70, Ku80, a combination thereof or a functional fragment thereof is transiently transfected into the cell.

In one embodiment there is provided a method as described herein, wherein the polynucleotide sequence encoding Ku70, Ku80, a combination thereof or a functional fragment thereof is transfected into the cell in the absence of screening or positive selection.

In one embodiment there is provided a method as described herein, wherein the polynucleotide sequence encoding Ku70, Ku80, a combination thereof or a functional fragment thereof is not integrated into the host cell genome, for example under the control of a constitutive or inducible promoter, such as an inducible promoter.

In one embodiment there is provided a method as described herein, wherein the polynucleotide sequence encoding Ku70, Ku80, a combination thereof or a functional fragment thereof is stably transfected into the cell (i.e. integrated into the host cell genome), for example under the control of a constitutive or inducible promoter, such as an inducible promoter.

In one embodiment there is provided a method as described herein wherein the DNA sequence encoding a protein of interest is stably integrated into the genome of the cell.

Transfected host cells of the present invention may be cultured in any suitable medium to produce the protein of interest and clones expressing suitable levels of protein selected.

As used herein, the term “comprising” in context of the present specification should be interpreted as “including”.

Embodiments and preferences may be combined as technically appropriate.

The disclosure herein describes embodiments comprising certain integers. The disclosure also extends to the same embodiments consisting or consisting essentially of said integers.

### EXAMPLE

Examples are based upon using either the CHOK1 or CHO-DG44 cell lines. These cells were transiently transfected with circular DNA encoding human Ku70 or Ku80 under the control of the CMV promoter in the presence of linear DNA encoding a protein of interest (in this case a MAb) which also contains a selection marker (in the case of MAb1-4 it is GS; and for MAb6-7 it is DHFR).

### Method for MAb1

CHOK1 cells were transiently transfected with circular DNA encoding human Ku70 or Ku80, under the control of the CMV promoter in the presence of linear DNA encoding a protein of interest and the GS selection marker.

### Method of Transfection

1×10⁷ cells were resuspended in 600 μl of CD-CHO culture media.

20 μg of linearised DNA (MAb+selection marker) with 20 μg of empty vector or 20 μg of Ku70 or Ku80 or 10 μg of both Ku70 and Ku80 were added to the cells so that a final volume of 800 μl was obtained (Table 1).

TABLE 1
Concentrations of MAb: Ku DNA added to the transfections.
Transfected DNA

| | | | | | Empty |
|---|---|---|---|---|---|
| | | MAb | Ku70 | Ku80 | vector |
| Experiment | Mix 1 | 20 μg | 0 | 0 | 20 μg |
| No. | Mix 2 | 20 μg | | 20 μg | 0 |
| | Mix 3 | 20 μg | 20 μg | | 0 |
| | Mix 4 | 20 μg | 10 μg | 10 μg | 0 |

The cells were electroporated using the BioRAD electroporator (conditions 300V, 15 ms, 1 pulse) and resuspended into 50 ml of CDCHO media supplemented with 2 mM Glutamine and incubated (37° C., 8% CO₂, 140 rpm) overnight.

### Limiting Dilution

The cells were counted and diluted into CDCHO media containing 25 μM MSX and these cells were plated into 96 well plates at 2500 cells per well. The cells were incubated (37° C., 8% CO₂) for 4 weeks.

4 weeks post transfection colonies that had grown were analysed for MAb expression using either Protein A or Protein G (Octet) (FIG. 1A). These were then transferred to 24 well plates where a 10 day culture was carried out and MAb expression was analysed using Protein A (FIG. 1B)

The above analysis was repeated at 6 weeks post transfection (FIGS. 2A and 2B).

### Method for MAb2

CHOK1 cells were transiently transfected with circular DNA encoding human Ku70 or Ku80 under the control of the CMV promoter in the presence of linear DNA encoding a protein of interest and the GS selection marker.

### Method of Transfection

1×10⁷ cells were resuspended in 600 μl of CD-CHO culture media.

20 μg of linearised DNA (MAb+selection marker) with 20 μg of empty vector or 20 μg of Ku70 or Ku80 or 10 μg each of both Ku70 and Ku80 were added to the cells so that a final volume of 800 μl was obtained (Table 2).

TABLE 2
Concentrations of MAb: Ku DNA added to the transfections.
Transfected DNA

| | | | | | Empty |
|---|---|---|---|---|---|
| | | MAb | Ku70 | Ku80 | vector |
| Experiment | Mix 1 | 20 μg | 0 | 0 | 20 μg |
| No. | Mix 2 | 20 μg | | 20 μg | 0 |
| | Mix 3 | 20 μg | 20 μg | | 0 |
| | Mix 4 | 20 μg | 10 μg | 10 μg | 0 |

The cells were electroporated using the BioRAD electroporator (conditions 300V, 15 ms, 1 pulse) and resuspended into 50 ml of CD-CHO media supplemented with 2 mM Glutamine and incubated (37° C., 8% CO₂, 140 rpm) overnight.

### Limiting Dilution

The cells were counted and diluted into CDCHO media containing 25 μMMSX and these cells were plated into 96 well plates at 2500 cells per well. The cells were incubated (37° C., 8% CO₂) for 4 weeks.

4 weeks post transfection colonies that had grown were analysed for MAb expression using either Protein A or Protein G (Octet) (FIG. 3A). These were then transferred to 24 well plates where a 10 day culture was carried out and MAb expression was analysed using Protein A (FIG. 3B)

### Pooled Stable Cell Line

The cells were counted and washed in PBS and resuspended at 5×10⁵ cells/ml in CD-CHO media containing 25 μM MSX in a final volume of 40 ml.

7 days post transfection the cells were counted and centrifuged and resuspended in fresh CD-CHO media containing 25 uM MSX so that the cells were at a density of 3×10⁵ cells/ml.

14 days post transfection or when the cells were >75% viable the cells were expanded into 25 ml shake flasks.

17 days post transfection or when the cells were >90% viable the cells were counted and split to 3×10̂5 cells/ml in 50 ml.

The cells were assayed for MAb expression using Protein A or G (Octet) (FIG. 4).

### Methods for MAb3

CHOK1 cells were transiently transfected with circular DNA encoding human Ku70 or Ku80 under the control of the CMV promoter in the presence of linear DNA encoding a protein of interest and the GS selection marker.

### Method of Transfection

1×10⁷ cells were resuspended in 600 μl of CD-CHO culture media.

20 μg of linearised DNA (MAb+selection marker) with 20 μg of empty vector or 20 μg of Ku70 or Ku80 or 10 μg each of both Ku70 and Ku80 were added to the cells so that a final volume of 800 μl was obtained (Table 3).

TABLE 3
Concentrations of MAb: Ku DNA added to the transfections
using electroporation only (A) or electroporation followed
by lipofectamine2000 (B).
Transfected DNA via electroporation
A

| | | | | | Empty |
|---|---|---|---|---|---|
| | | MAb | Ku70 | Ku80 | vector |
| Experiment | Mix 1 | 20 μg | 0 | 0 | 20 μg |
| No. | Mix 2 | 20 μg | 10 μg | 10 μg | 0 |

B

| | | | | Empty | MAb by |
|---|---|---|---|---|---|
| | | Ku70 | Ku80 | vector | lipo |
| Experiment | Mix 1 | 0 | 0 | 40 μg | 20 ug |
| No. | Mix 2 | 20 μg | 20 μg | 0 | 20 ug |

The cells were electroporated using either the BioRAD electroporator (conditions 300V, 15 ms, 1 pulse) and resuspended into 50 ml of CDCHO media supplemented with 2 mM Glutamine and incubated (37° C., 8% CO₂, 140 rpm) overnight (Table 3A) or they were electroporated and subsequently exposed to a lipid based transfection method using lipofectaime 2000 according to the manufacturers' instructions (1×10⁷ cells were transfected with 20 ug of MAb DNA) to introduce the MAb DNA and then incubated overnight (Table 3B).

### Limiting Dilution

The cells were counted and diluted into CDCHO media containing 25 μM MSX and these cells were plated into 96 well plates at 2500 cells per well. The cells were incubated (37° C., 8% CO₂) for 4 weeks.

All growing transfectants were expanded up to the 24 well stage cultures and were analysed for MAb expression using Protein A or Protein G (Octet) after 10 days of growth (FIG. 5A)

In some cases the top transfectants were transferred to 6 well plates and on into 25 ml and were then assessed for MAb expression using Protein A or Protein G (Octet) after 12 days of growth (FIG. 5B).

The number of expressing antibody transfectants were analysed for Mab1, 2 &3. (E=electroporation; E+Lipo=electroporation+lipofectamine2000). 12-21% more expressing transfectants were obtained when co-transfected with Ku proteins compared to the control when using the GS selection system (FIG. 6). Transfection of nucleotide sequences encoding protein factors involved in the normal cellular process of ‘non-homologous end joining’ such as the human genes Ku70 and Ku80 at the same time as an expression cassette for a protein of interest has been found to increase the efficiency of the process of making stably integrated expressing cell lines. The increased efficiency was evidenced in a number of useful ways such as increased numbers of transfectant expressing and/or transfectant expressing higher levels of the protein of interest were observed. These effects can be useful by reducing the amount of effort and time required to find a transfectant expressing a target yield or increasing the maximum product yield.

### Methods for MAb4

CHO-DG44 cells were transiently transfected with circular DNA encoding human Ku70 or Ku80 under the control of the CMV promoter in the presence of linear DNA encoding a protein of interest and the DHFR selection marker

### Method of Transfection

A total of 1×10⁷ cells were nucleofected at a concentration of 1×10⁶ cells+10 ug of DNA per cuvette using nucleofection with the Amaxa electroporator. The cells were nucleofected using solution L (conditions U) and resuspended into 40 ml of DG44 medium supplemented with 8 mM Glutamine and incubated (37° C., 8% CO₂) overnight.

TABLE 4
Concentrations of MAb: Ku DNA added to
the transfections using nucleofection.
Transfected DNA via nucleofection of 1 × 10⁷ cells

| | | | | Empty | |
|---|---|---|---|---|---|
| | | Ku70 | Ku80 | vector | MAb |
| Experiment | Mix 1 | 0 | 0 | 50 μg | 50 ug |
| No. | Mix 2 | 25 μg | 25 μg | 0 | 50 ug |

### Limiting Dilution

Cells were plated at 2000, 4000, or 4000+5 or 10 nm MTX per well in CD-CHO media with the addition of 8 mM Glutamine.

2-3 weeks post transfection colonies with >10% confluency were picked from the plates containing 10 nM MTX or other transfectant were discarded. These transfectant were moved to 24 well plates and assayed for expression based upon binding rate, the top 24 transfectant were then moved to 6 well and then onto 30 ml cultures for pictogram/cell/day values to be obtained (FIG. 7a). The top 6 transfectants from each condition based upon picograms per cell per day (PCD) were grown in 100 ml of production media for 14 days and expression levels obtained (FIG. 7b).

### Methods for MAb5

CHO-DG44 cells were transiently transfected with circular DNA encoding human Ku70 or Ku80 under the control of the CMV promoter in the presence of linear DNA encoding a protein of interest and the DHFR selection marker

### Method of Transfection

1×10⁷ cells were resuspended in 600 μl of CD-CHO culture media.

20 μg of linearised DNA (MAb+selection marker) with 20 μg of empty vector or 20 μg of Ku70 or Ku80 or 10 μg each of both Ku70 and Ku80 were added to the cells so that a final volume of 800 μl was obtained (Table 3A).

The cells were electroporated using either the BioRAD electroporator (conditions 300V, 15 ms, 1 pulse) and resuspended into 50 ml of CDCHO media supplemented with 2 mM Glutamine and incubated (37° C., 8% CO₂, 140 rpm) overnight.

### Limiting Dilution

The cells were counted and plated at 2000, 4000, or 4000+5, 10 nm or 15 nm MTX per well in CDCHO media with the addition of 8 mM Glutamine.

All growing transfectants were expanded up to the 24 well stage cultures and were analysed for MAb expression using Protein A or Protein G (Octet) after 12 days of growth (FIG. 8).

### FIGURES

FIG. 1A Shows expression analysis of clones (transfectants) expressing a MAb1 (in an IgG4 format) obtained from 96 well plates 4 weeks post transfection. Expression levels were analysed using Protein A Octet. MAb expressing transfectants were obtained following a co-transfection of CHOK1 cells with the transient expression of Ku protein(s), and the stable integration of MAb1 heavy chain and light chain DNA with a selection marker. 4 weeks post transfection all growing transfectants were analysed for MAb expression level.

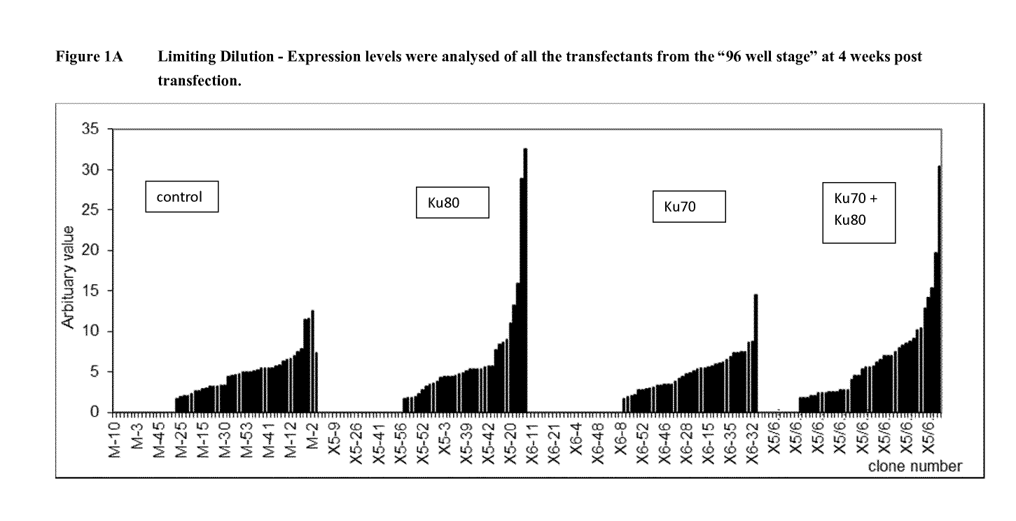

FIG. 1B All clones (transfectants) analysed at 96 well stage were expanded to 24 well stage and analysed for expression of MAb1, 10 days post seeding, using Protein A Octet.

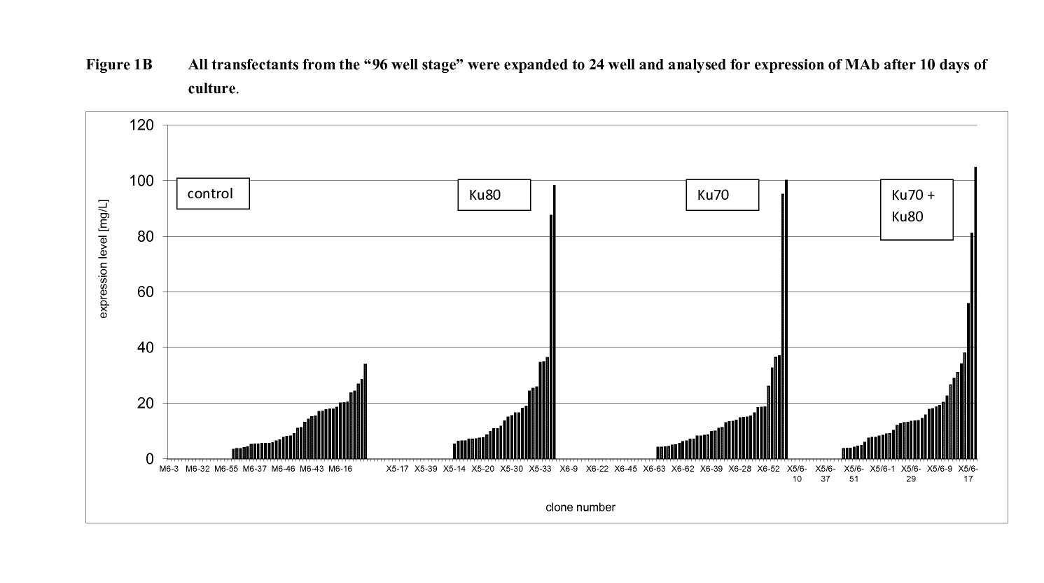

FIG. 2A Shows expression analysis of 24 randomly selected transfectants obtained from 96 well plates 6 weeks post transfection and analysed using Protein A Octet. MAb1 expressing transfectants were obtained following a co-transfection of CHOK1 cells with Ku protein(s), MAb1 heavy chain and light chain DNA with GS selection. An additional 61, 102, 102 and 82 transfectants for the control, Ku80, Ku70 and Ku80+Ku70 transfections respectively grew between 4-6 weeks post transfection. 24 transfectants of, approximately the same size, from each experiment were randomly selected and analysed for MAb1 expression level using Protein A Octet. No Statistical difference between the mean expression level of the control transfectants and those transfected with either Ku70 and Ku80 protein—Mann Whitney (T-test) p<0.05 ns. Statistical difference between the mean expression level of the control transfectants and those co-transfected with Ku70 and Ku80 protein—Mann Whitney (T-test) p<0.05*

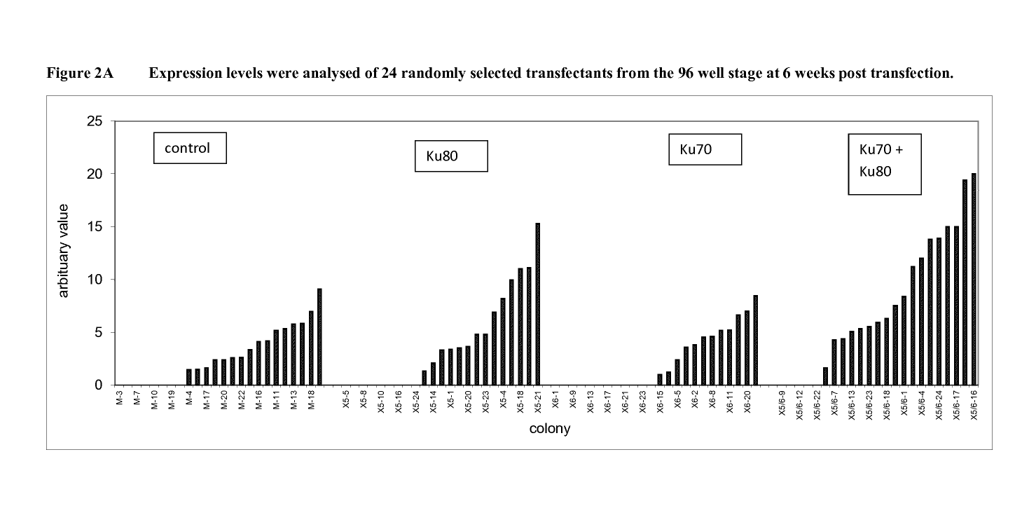

FIG. 2B Shows expression analysis of 24 randomly selected transfectants expressing a MAb1 (in an IgG4 format) expanded from 96 well plates to 24 well plates and cultured for 12 days prior to expression analysis. Expression levels were analysed using Protein A Octet.

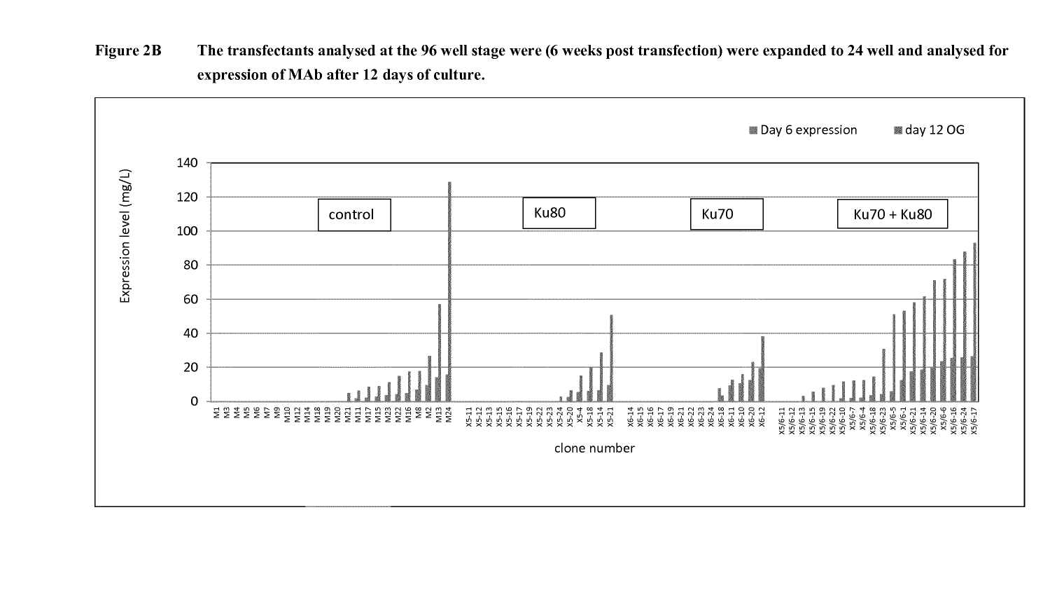

FIG. 3A Shows expression analysis of clones (transfectants) expressing MAb2 (in an IgG1 format) were obtained from 96 well plates 4 weeks post transfection. Expression levels were analysed using Protein G Octet. MAb2 expressing transfectants were obtained following a co-transfection of CHOK1 cells with the transient expression of Ku protein(s), and the stable integration of MAb2 heavy chain and light chain DNA with a GS selection marker. 4 weeks post transfection all growing transfectants were analysed for MAb expression level. Statistically significant differences between the mean expression level of the control transfectants and Ku protein transfectants were obtained—Mann Whitney (T-test) p<0.005***

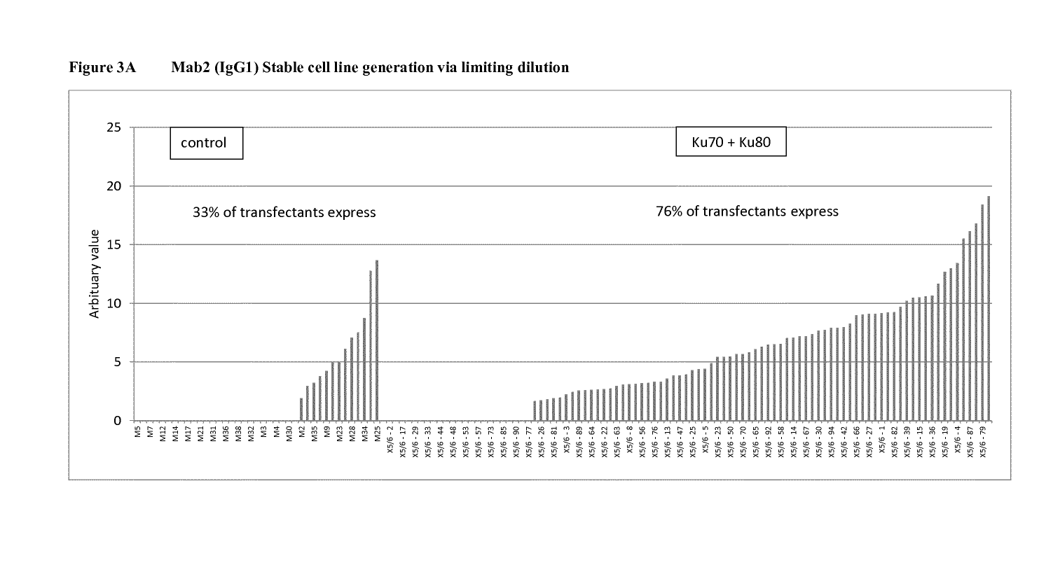

FIG. 3B Expression analysis of transfectants expressing a MAb2 (IgG1) were expanded from 96 well plates to 24 well plates and grown for 10 days prior to expression analysis. Expression levels were analysed using Protein G Octet. Statistically significant differences between the mean expression level of the control transfectants and Ku protein transfectants were obtained—Mann Whitney (T-test) p<0.005***

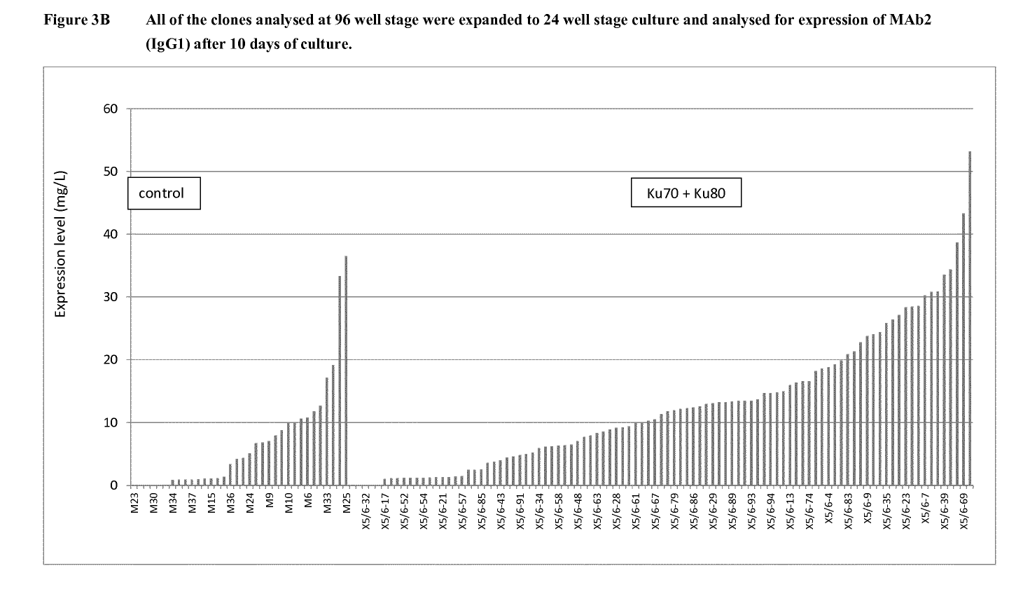

FIG. 4 Shows expression analysis of MAb2 (in an IgG1 format) using a pooled stable approach.

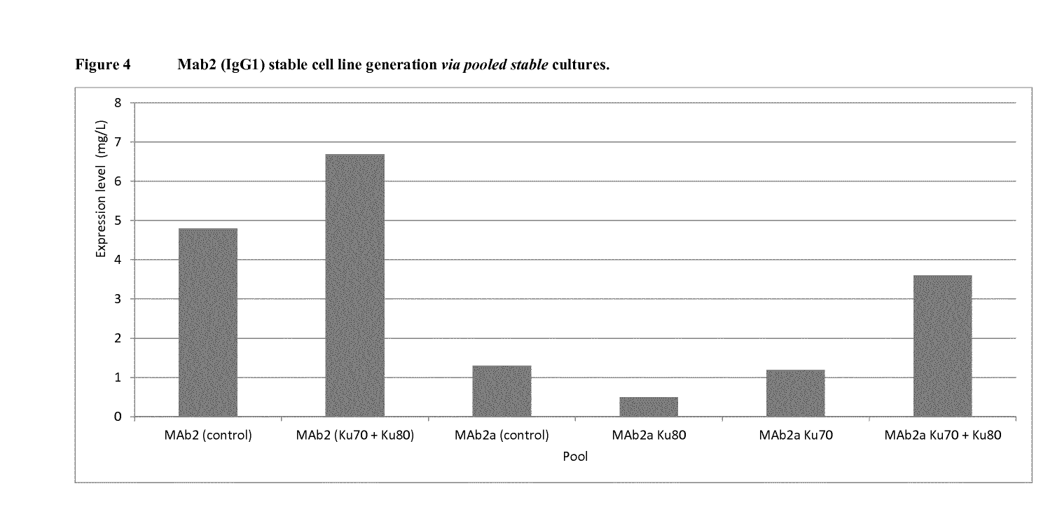

MAb2 expressing pooled stables were obtained following a co-transfection of CHOK1 cells with the transient expression of the Ku protein(s), MAb2 HC and LC DNA with GS selection marker. The cells were left to recover and once >90% viable were seeded into spin tubes and expression analysis was carried out at day 5 after seeding using Protein G Octet.

The addition of Ku70 and Ku80 at the time of transfection resulted in a 1.4-2.7-fold increase in the expression level of the pool.

FIG. 5A Shows expression analysis of transfectants expressing MAb3 obtained from 96 well plates and transferred to the 24 well stage, following a co-transfection of CHOK1 cells with the transient expression of Ku protein(s), and the stable integration of MAb3 heavy chain and light chain DNA with a GS selection marker using either electroporation or a combination of electroporation and Lipofectaime 2000 (Life Technologies).

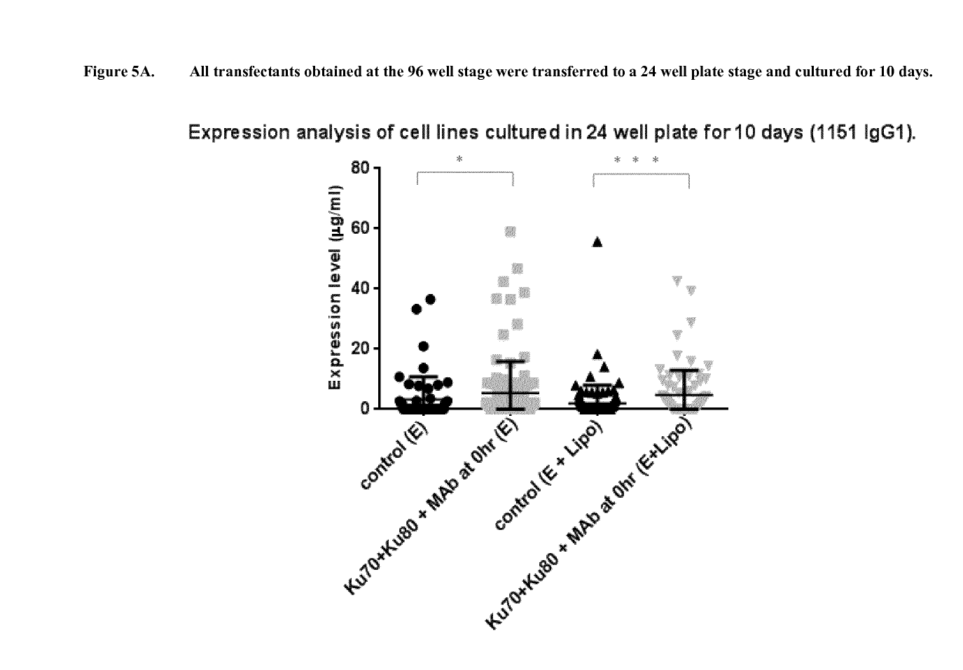

4 weeks post transfection all growing transfectants were analysed for MAb expression level using Protein G Octet.

FIG. 5B Shows expression analysis of the “top” 6 trasnfectants at the 24 well stage cultured in a 24 well plate for 12 days. The top 6 control and Ku transfected transfectants obtained from the electroporation method were transferred to 25 ml of media and cultured for 12 days and expression levels were assayed using protein G Octet. (C=control; K=Ku).

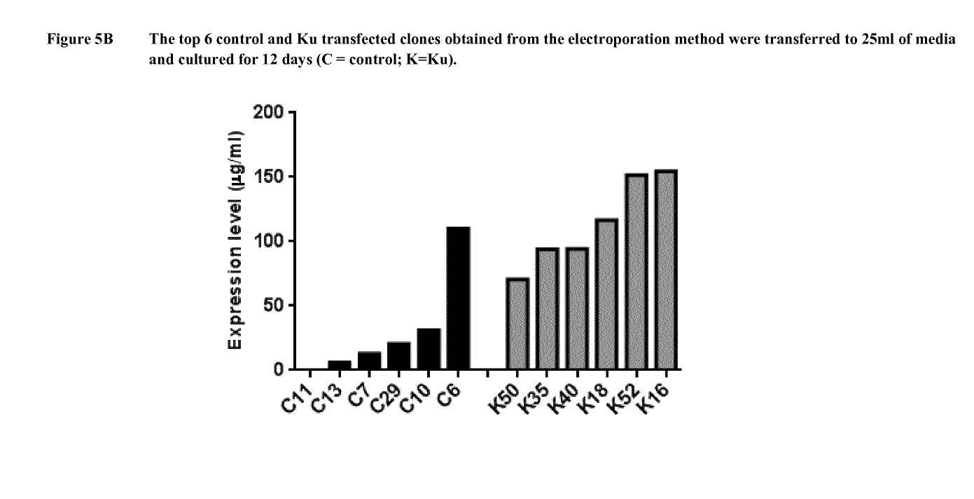

FIG. 6. The number of expressing antibody transfectants were analysed for Mab1, 2 &3. (E=electroporation; E+Lipo=electroporation+lipofectamine2000). 12-21% more expressing transfectants were obtained when co-transfected with Ku proteins compared to the control when using the GS selection system. (C=control; K=Ku).

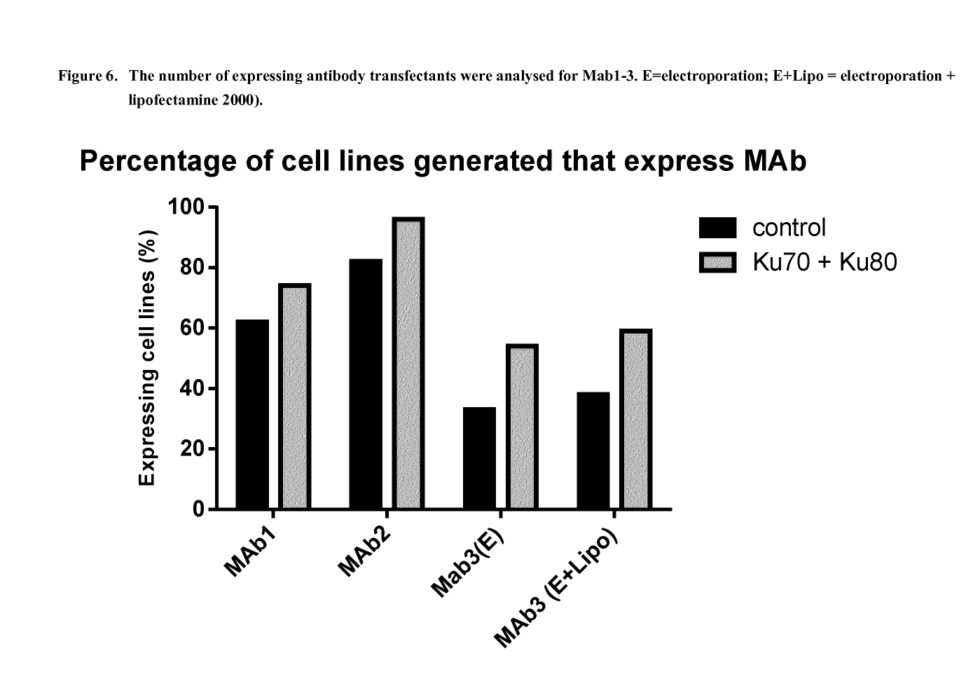

FIG. 7A Mab 4 employed the DHFR selection system, clones with >10% confluency which grew on the highest concentration of MTX (10 nM) were transferred to 24 and then 6 well plates before being assayed for PCD in 25 ml cultures. The top 24 control or Ku transfection clones were ranked based upon binding rate in 24 well plates had their PCD calculated. (C=control; K=Ku).

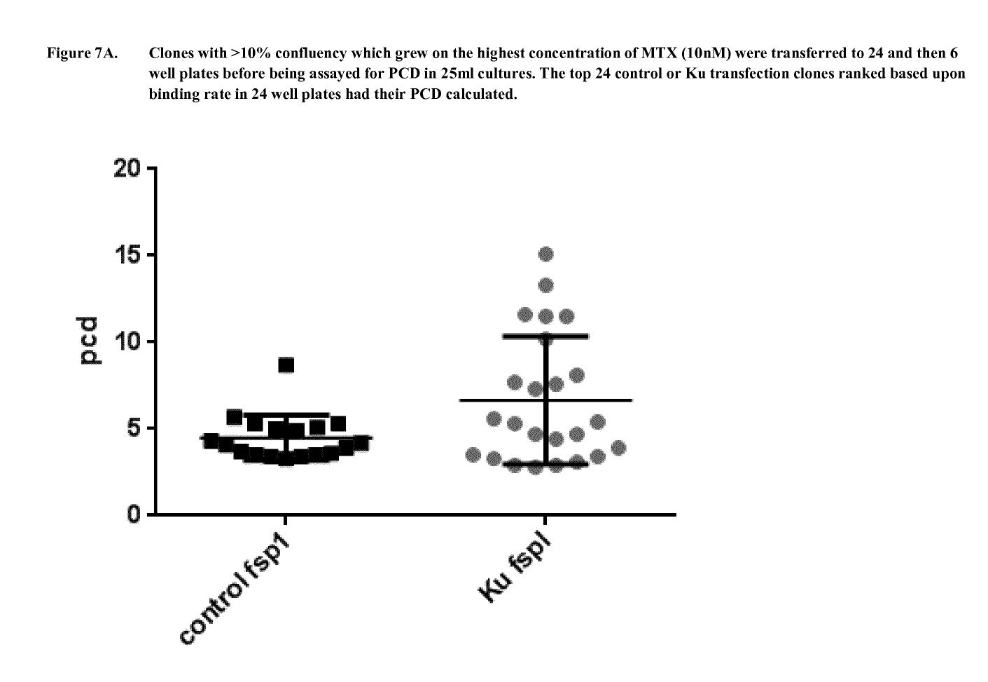

FIG. 7B The top 6 clones based upon PCD at 25 ml scale were grown in 100 ml of production media for 14 days and their final expression levels were analysed using Protein G Octet at the end of the culture period. (C=control; K=Ku).

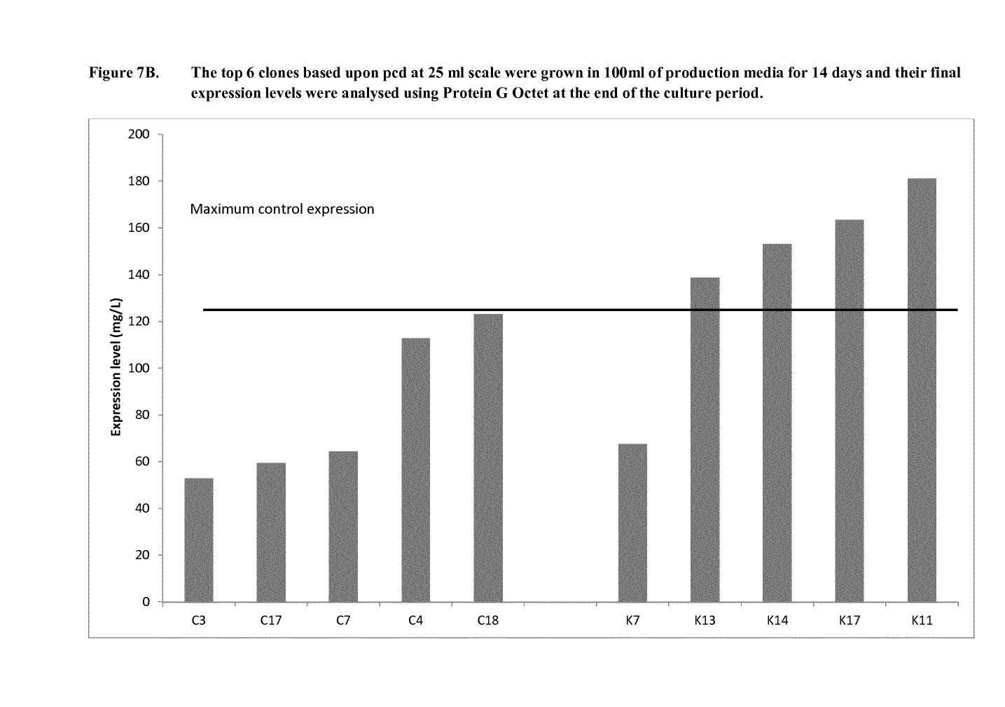

FIG. 8 MAb5 was prepared employing the DHFR selection system. All transfectants that grew at the 96 well plate stage were transferred to the 24 well stage and cultured for 12 days. Expression analysis was carried out using Protein G Octet.

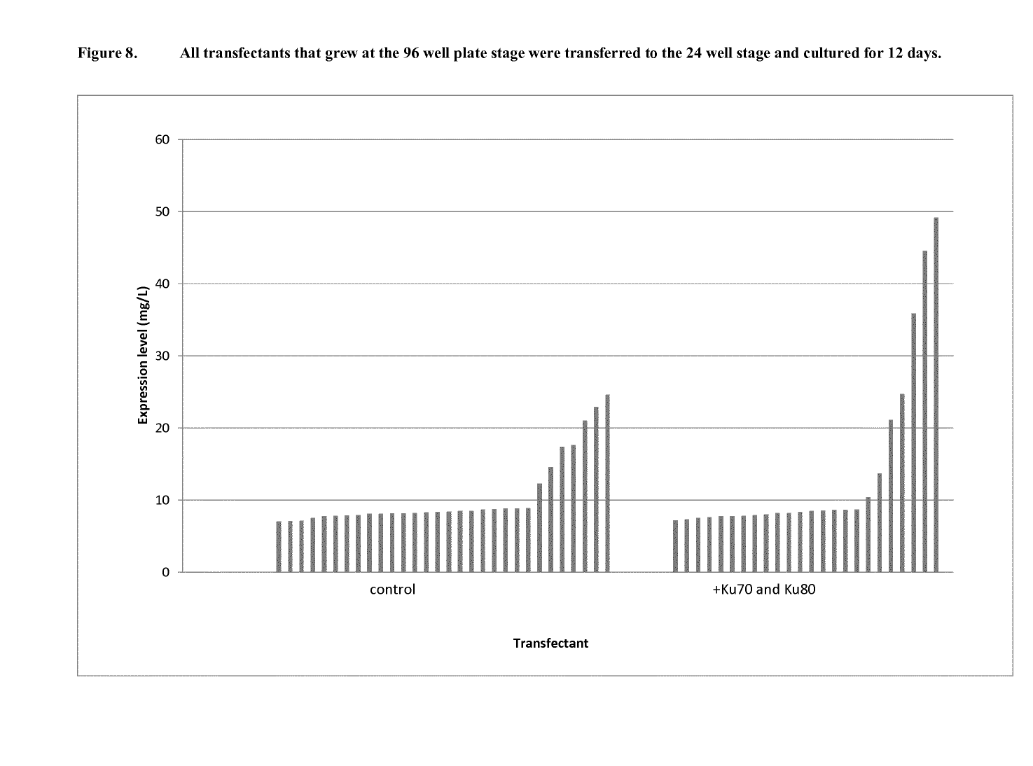

1. A host cell transfected with a NHEJ protein or a functional fragment thereof, or a polynucleotide encoding the NHEJ protein or a functional fragment thereof wherein said polynucleotide sequence is under the control of a suitable promoter and the host cell is also transfected with an expression cassette comprising a polynucleotide (DNA or RNA) sequence encoding at least one protein of interest.

2. The host cell according to claim 1, where the host cell is transfected with a polynucleotide sequence encoding an NHEJ protein or a functional fragment thereof, wherein said polynucleotide sequence is under the control of a suitable promoter and the host cell is also transfected with an expression cassette comprising a polynucleotide sequence encoding at least one protein of interest.

3. The host cell according to claim 1 wherein the NHEJ protein is Ku70 or Ku80 or a functional fragment thereof.

4. The host cell according to claim 1 transfected with two or more NHEJ proteins or functional fragments thereof or a polynucleotide or polynucleotides encoding the same.

5. The host cell according to claim 1 wherein the host cell is transfected with Ku70 and Ku80 or functional fragments thereof or a polynucleotide or polynucleotides encoding the same.

6. The host cell according to claim 1, wherein the cell is a prokaryotic cell, a eukaryotic cell or a yeast.

7. The host cell according to claim 6 wherein the host cell is a CHO cell.

8. The host cell according to claim 1, wherein the protein of interest is an antibody or binding fragment thereof.

9. The host cell according to claim 1, wherein the protein of interest is a protein causing site specific mutagenesis such as a zinc finger nuclease, mega-nuclease or functional fragments thereof.

10. The host cell according to claim 1, wherein the polynucleotide is DNA.

11. The host cell according to claim 1, wherein the polynucleotide is RNA.

12. The host cell according to claim 1, wherein the polynucleotide sequence encoding the NHEJ protein is in the form of a plasmid.

13. The host cell according to claim 1, wherein the polynucleotide sequence encoding a protein of interest is in the form of a plasmid.

14. The host cell according to claim 1, wherein the polynucleotide sequence encoding a protein of interest is in the form of linear DNA or RNA

15. The host cell according to claim 1, wherein the DNA sequence encoding a protein of interest is provided in the form of a plasmid and wherein the plasmid further comprises the DNA sequence encoding Ku70, Ku80, a combination of Ku70 and Ku80 or a functional fragment thereof.

16. The host cell according to claim 1, wherein the promoter is a strong viral promoter.

17. The host cell according to claim 16, wherein the promoter is a CMV promoter.

18. The host cell according to claim 1, wherein the NHEJ protein or functional fragment thereof is of human, hamster, mouse, yeast or other sequence origin.

19. The host cell according to claim 1, wherein the polynucleotide sequence encoding the NHEJ protein or a functional fragment thereof and/or the expression cassette comprising a DNA sequence encoding a protein of interest further comprise a selection marker.

20. The host cell according to claim 19, wherein the selection marker is a glutamine synthetase marker.

21. The host cell according to claim 1, wherein the expression cassette comprising a DNA sequence encoding a protein of interest is stably integrated into the genome of the cell.

22. The host cell according to claim 1, wherein the polynucleotide sequence encoding the NHEJ protein or a functional fragment thereof is transiently transfected into the cell.

23. The host cell according to claim 1, wherein the polynucleotide sequence encoding the NHEJ protein or a functional fragment thereof is stably transfected into the cell.

24. A plasmid DNA comprising a sequence encoding Ku70, Ku80, a combination thereof or a functional fragment thereof wherein said DNA sequence is under the control of a suitable promoter.

25. The plasmid DNA according to claim 24, wherein the plasmid further comprises a DNA encoding a protein of interest, for example wherein the protein of interest is an antibody or a binding fragment thereof.

26. The plasmid DNA according to claim 24, wherein the DNA is circular.

27. The plasmid DNA according to claim 24, wherein the DNA is linear.

28. A method of generating a host cell capable of expressing a protein of interest encoded by a polynucleotide sequence comprising the step of transfecting the cell with:
a) Ku70, Ku80, a combination thereof or a functional fragment thereof, or a polynucleotide sequence or sequences encoding same wherein said polynucleotide sequence is under the control of a suitable promoter, and
b) an expression cassette comprising a polynucleotide (DNA or RNA) sequence encoding a protein of interest.

29. The method according to claim 28, wherein the transfection of the components in step a) and b) is co-transfection (concomitant transfection).

30. The method according to claim 28, wherein the transfection of the components in step a) takes place before the components in step b).

31. The method according to claim 28, wherein the transfection of the components in step b) takes place before the components in step c).

32. The method according to claim 28 wherein the polynucleotide sequence in step a) is a DNA sequence encoding Ku70, Ku80, a combination thereof or a functional fragment thereof.

33. The method according to claim 28 wherein the polynucleotide sequences in step a) are a DNA sequence encoding Ku70 and a DNA sequence encoding Ku80.

34. The method according to claim 32 where the polynucleotide sequence is provided in the form of a plasmid.

35. The method according to claim 34, wherein the plasmid is defined in claims 24 to 27.

36. The method according to claim 28, wherein the expression cassette comprising a DNA sequence encoding a protein of interest is provided on a plasmid.

37. The method according to claim 28, wherein the polynucleotide sequence encoding Ku70, Ku80, a combination thereof or a functional fragment thereof is transiently transfected into the cell.

38. The method according to claim 28, wherein the DNA sequence encoding a protein of interest is stably integrated into the genome of the cell.
